(19) United States
(12) Patent Application Publication
(10) Pub. No.: US 2011/0306641 A1
(43) Pub. Date: Dec. 15, 2011

(54) NOVEL 2,6-SUBSTITUTED-3-NITROPYRIDINE DERIVATIVE, METHOD FOR PREPARING SAME, AND PHARMACEUTICAL PREPARATION INCLUDING SAME

(76) Inventors: Jei Man Ryu, Gyeonggi-do (KR); Jin Soo Lee, Gyeonggi-do (KR); Whui Jung Park, Gyeonggi-do (KR); Yun Ha Hwang, Gyeonggi-do (KR); Ki_Yoon Kim, Gyeonggi-do (KR)

(21) Appl. No.: 13/133,649

(22) PCT Filed: Dec. 4, 2009
(86) PCT No.: PCT/KR2009/007218

§ 371 Date: Aug. 25, 2011

(30) Foreign Application Priority Data
Dec. 10, 2008 (KR) 10-2008-0125362

(51) Int. Cl. A61K 31/4439; A61P 19/10; C07D 401/14

(52) U.S. Cl. 514/338; 546/272.7

(57) ABSTRACT

The present invention relates to a novel 2,6-substituted-3-nitropyridine derivative compound, a method for preparing same, a pharmaceutical composition for prevention and treatment of osteoporosis including same, applications of same for preparing the therapeutic agent of osteoporosis prevention and treatment, and a method for prevention and treatment of osteoporosis using same. The 2,6-substituted-3-nitropyridine derivative compound of the present invention effectively increases osteoblast activity and also inhibits the formation of osteoclasts, so that it may be usefully used for the prevention and treatment of osteoporosis.

### TECHNICAL FIELD

The present invention relates to a novel 2,6-substituted-3-nitropyridine derivative compound, a method for preparing the same, a pharmaceutical composition containing the same, a method for the prevention and treatment of osteoporosis using the same, and use of the same for manufacturing an anti-osteoporotic agent.

### BACKGROUND ART

Bone is a supporting material for the body's framework and serves to conserve the necessary bone mass and structure. Bone also functions as a reservoir of calcium (Ca²⁺) or the like and plays an important role in maintaining blood levels of calcium or the like. To cope with these functions, the growth of bone is a metabolic balance between the activity of osteoblasts and osteoclasts in the bone remodeling cycle. Accordingly, bone is in a steady state, which maintains good balance between bone absorption and bone formation in the process of metabolism by continuously performing both bone absorption and bone formation. When the balance between bone absorption and bone formation is disrupted, the degree of bone absorption is relatively higher than that of bone formation, which may lead to osteoporosis, a condition which causes reduction in bone density or bone mass, resulting in decrease in bone strength. This is a disease which frequently occurs in middle-aged or elderly women.

Osteoporosis is a disease, which results from a disturbance in the balance between bone absorption and bone formation, and is caused by having a higher degree of bone absorption relative to that of bone formation. Osteoporosis reduces calcification of bone tissues, and decreases the level of the compact substances in the bone, which broadens the marrow cavity. As osteoporosis progresses, bone becomes brittle, and bone fracture may easily occur even with a small impact. Bone is a steady state structure, in which the bone formation by osteoblasts and the bone resorption by osteoclasts occur continuously.

Previous studies on osteoporosis have focused mainly on dysmetabolism of bone minerals such as calcium and phosphorus. However, such studies did not provide sufficient findings on the pathogenic mechanism of osteoporosis.

Although bisphosphonate (alendronate, etidronate, etc.), hormone therapy (raloxifene), vitamin D, calcitonin, calcium agents, and the like have been currently used as an anti-osteoporotic agent, they are known to have adverse side effects. Specifically, bisphosphonate agents exhibit low absorptivity, difficulty of administration and risk of causing esophagitis. Hormone agents must be administered throughout a patient's life and long-term administration thereof may result in adverse side effects such as breast cancer, uterus cancer, gallstones and thrombosis. Vitamin D agents are expensive and show little efficacy, and calcitonin agents are also very expensive and have difficulty of administration. Calcium agents have few adverse side effects, but their medicinal effects are disadvantageously restricted to the prevention of osteoporosis, not the treatment thereof.

Osteoporosis cannot be treated with short-term administration of drugs and generally requires long-term administration of drugs. Therefore, there is a need for a novel substance having excellent medicinal efficacy without causing the above-mentioned adverse side effects even upon long-term administration thereof.

### DISCLOSURE OF THE INVENTION

### Technical Problem

For the purpose of finding an effective therapeutic agent against osteoporosis, the inventors of the present invention have established a screening method capable of evaluating effects of various substances on the differentiation and activity of osteoclasts and osteoblasts and carried out the evaluation of such substances. As a result, the present inventors have discovered that novel 2,6-substituted-3-nitropyridine derivative compounds and particularly 3-nitropyridine derivatives having anti-proliferative effects on Hepatitis B virus (HBV) and Human Immunodeficiency Virus (HIV) disclosed in Korean Patent Application Nos. 10-1999-0064403 and 10-1999-0053295, both of which were assigned to the present applicant, not only suppress the differentiation into osteoclasts (formation of osteoclasts or osteoclastogenesis) to effectively inhibit osteoclastic bone absorption, but also promotes the activity of osteoblasts to thereby effectively increase osteogenesis. The present invention has been completed based on these findings.

Therefore, the present invention is intended to provide a novel 2,6-substituted-3-nitropyridine derivative compound, a method for preparing the same, and a composition for the prevention or treatment of osteoporosis, containing an effective amount of a 2,6-substituted-3-nitropyridine derivative represented by formula 1, including the same, or a salt thereof.

### Technical Solution

The present invention provides the following novel 2,6-substituted-3-nitropyridine derivative compounds or pharmaceutically acceptable salts thereof:

Compound 1: 2-(1H-indazol-5-yl)amino-6-(2-methyl-4,5-dihydroimidazol-1-yl)amino-3-nitropyridine,

Compound 2: 2-(1H-indazol-5-yl)amino-6-(2-methylimidazol-1-yl)-3-nitropyridine,

Compound 3: 2-(1H-indazol-5-yl)amino-6-(2-isopropylimidazol-1-yl)-3-nitropyridine, and

Compound 4: 2-(1H-indazol-5-yl)amino-6-(5-methylimidazol-1-yl)-3-nitropyridine.

As used herein, the term “pharmaceutically acceptable salt” refers to inorganic or organic acid salts which are generally used for the preparation of pharmaceutical products by pharmaceutical manufacturers. Examples of the inorganic acid include hydrochloric acid, hydrobromic acid, sulfuric acid, and phosphoric acid. Examples of the organic acid include citric acid, acetic acid, lactic acid, tartaric acid, fumaric acid, formic acid, propionic acid, oxalic acid, trifluoroacetic acid, methanesulfonic acid, benzenesulfonic acid, maleic acid, benzoic acid, gluconic acid, glycolic acid, succinic acid, 4-morpholineethanesulfonic acid, camphorsulfonic acid, 4-nitrobenzenesulfonic acid, hydroxy-O-sulfonic acid, 4-toluenesulfonic acid, galacturonic acid, embonic acid, glutamic acid, and aspartic acid. Preferably, the inorganic acid is hydrochloric acid, and the organic acid is methanesulfonic acid.

Further, the present invention provides a method for preparing a 2,6-substituted-3-nitropyridine derivative compound of formula 1, which includes the following steps:

a) a step of reacting 2,6-dichloro-3-nitropyridine with 5-aminoindazole in the presence of a base to prepare a 2-(1H-indazol-5-yl)amino-6-chloro-3-nitropyridine; and

b) a step of reacting 2-(1H-indazol-5-yl)amino-6-chloro-3-nitropyridine prepared in Step a) with an amine compound selected from 2-methyl-2-imidazoline, 2-methylimidazole, 2-isopropylimidazole and 5-methylimidazole to prepare a 2,6-substituted-3-nitropyridine derivative compound in which the amine compound is introduced at the 6-position of the pyridine ring.

In the preparation method of the present invention, 2,6-dichloro-3-nitropyridine and 5-aminoindazole used as starting materials of Step a) are easily commercially available or may be prepared by a known method in the art.

In the preparation method of the present invention, the “base” of Step a) may be appropriately selected and used from various organic bases, preferably tertiary organic bases. For example, the base is preferably at least one selected from triethylamine, N,N-diisopropylethylamine, N-methylmorpholine, N-methylpiperidine, 4-dimethylamino-pyridine, N,N-dimethylaniline, 2,6-lutidine and pyridine.

In Step a) or Step b) of the preparation method of the present invention, the reaction solvent is preferably selected from alcohols such as methanol, ethanol and isopropanol, acetonitrile, chloroform, methylene chloride, tetrahydrofuran, N,N-dimethylformamide, N-methylpyrrolidinone and any combination thereof.

Although the reaction temperature of Step b) may vary depending on the type of amine compound, it is preferably in the range of 25 to 80°.

Further, the present invention provides a composition for the prevention or treatment of osteoporosis, containing an effective amount of a 3-nitropyridine derivative compound represented by formula 1, including the compound of the present invention, or a salt thereof:

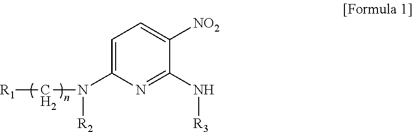

wherein:

R₁ represents H, hydroxy, amino, C₃-C₆ linear or branched aminoalkyl, C₂-C₆ dialkylamino, C₂-C₆ linear or branched hydroxyalkyl, C₃-C₆ linear or branched dihydroxyalkyl, C₁-C₃ alkoxy-substituted C₁-C₆ linear or branched alkyl, or a saturated or unsaturated 5- or 6-membered heterocyclic compound which contains 1 to 3 hetero atoms selected from N, O and S and is unsubstituted or substituted by a C₁-C₃ alkyl group, R₁ optionally contains an asymmetric carbon atom,

R₂ represents H, C₁-C₄ linear or branched alkyl, or unsubstituted or substituted C₃-C₆ cyclicalkyl, or

R₁ and R₂ taken together a saturated or unsaturated 5 or 6-membered heterocyclic compound wherein the heterocyclic ring contains 1 to 3 hetero atoms selected from N, O and S and is unsubstituted or substituted by one or more group selected from C₁-C₅ linear or branched alkyl, amino, C₂-C₅ linear or branched hydroxyalkyl and hydroxy,

R₃ represents indazol-5-yl or indazol-6-yl, and

n represents an integer of 0 to 4.

In the present invention, the compound of formula 1 is preferably a compound wherein R, represents H, an amino group, hydroxy, methoxy, pyridinyl, imidazolyl, 1,3-dioxalane, morpholine, unsubstituted or substituted piperazine, N-methylpiperazine, 3-dimethylamino-2,2-dimethylpropyl or NR₄R₅ wherein R₄ and R₅ each independently represent hydrogen, methyl, t-butyl, morpholinyl or N-methylpiperazine; R₂ represents H, methyl, ethyl, isopropyl, isobutyl, cyclic propyl, 2-amino cyclic hexyl or t-butyl; or R₁ and R₂ taken together form 4-hydroxypiperidin-1-yl, 4-aminopiperidin-1-yl, piperazin-1-yl, 4-methylpiperazin-1-yl, 3,5-dimethylpiperazin-1-yl, 2,2,6,6-tetramethylpiperazin-1-yl, 4-hydroxyethylpiperazin-1-yl, methylimidazoline, ethylimidazolyl or isopropylimidazolyl; R₃ represents indazol-5-yl or indazol-6-yl; and n represents an integer of 0 to 4.

Among the compounds of formula 1 in accordance with the present invention, preferable compounds are as follows:

Compound 1: 2-(1H-indazol-5-yl)amino-6-(2-methyl-4,5-dihydroimidazol-1-yl)-amino-3-nitropyridine,

Compound 2: 2-(1H-indazol-5-yl)amino-6-(2-methylimidazol-1-yl)-3-nitropyridine,

Compound 3: 2-(1H-indazol-5-yl)amino-6-(2-isopropylimidazol-1-yl)-3-nitropyridine,

Compound 4: 2-(1H-indazol-5-yl)amino-6-(5-methylimidazol-1-yl)-3-nitropyridine,

Compound 5: 2-(1H-indazol-5-yl)amino-6-(methylamino)-3-nitropyridine,

Compound 6: 2-(1H-indazol-5-yl)amino-6-(isopropylamino)-3-nitropyridine,

Compound 7: 2-(1H-indazol-5-yl)amino-6-[(N-methyl-2-hydroxyethyl)amino]-3-nitropyridine,

Compound 8: 2-(1H-indazol-5-yl)amino-6-(isobutylamino)-3-nitropyridine,

Compound 9: 2-(1H-indazol-5-yl)amino-6-(4-hydroxypiperidino)-3-nitropyridine,

Compound 10: 2-(1H-indazol-5-yl)amino-6-(piperazin-1-yl)-3-nitropyridine,

Compound 11: 2-(1H-indazol-5-yl)amino-6-[(N-ethyl-2-hydroxyethyl)amino]-3-nitropyridine,

Compound 12: 2-(1H-indazol-5-yl)amino-6-(t-butylamino)-3-nitropyridine,

Compound 13: 2-(1H-indazol-5-yl)amino-6-(2,2,6,6-tetramethylpiperazin-4-yl)-3-nitropyridine,

Compound 14: 2-(1H-indazol-6-yl)amino-6-[(2-pyridyl)ethylamino]-3-nitropyridine,

Compound 15: 2-(1H-indazol-5-yl)amino-6-[4-(2-hydroxyethyl)piperazin-1-yl]-3-nitropyridine,

Compound 16: 2-(1H-indazol-5-yl)amino-6-[(3-dimethylamino-2,2-dimethyl)-propylamino]-3-nitropyridine,

Compound 17: 2-(1H-indazol-5-yl)amino-6-[(N-[1,3]-dioxolan-2-ylmethyl)-methylamino]-3-nitropyridine,

Compound 18: 2-(1H-indazol-6-yl)amino-6-[(1-(S)-methyl-2-hydroxyethyl)-amino]-3-nitropyridine,

Compound 19: 2-(1H-indazol-6-yl)amino-6-[4-(2-hydroxyethyl)piperazin-1-yl]-3-nitropyridine,

Compound 20: 2-(1H-indazol-6-yl)amino-6-[2-(N,N-dimethylamino)ethylamino]-3-nitropyridine,

Compound 21: 2-(1H-indazol-6-yl)amino-6-[(2-pyridyl)methylamino]-3-nitropyridine,

Compound 22: 2-(1H-indazol-5-yl)amino-6-[(1-methyl-2-methoxy)ethylamino]-3-nitropyridine,

Compound 23: 2-(1H-indazol-6-yl)amino-6-[(4-methylpiperazin-1-yl)amino]-3-nitropyridine,

Compound 24: 2-(1H-indazol-6-yl)amino-6-[(4-morpholin-1-yl)amino]-3-nitropyridine,

Compound 25: 2-(1H-indazol-5-yl)amino-6-(N,N-dimethylamino)-3-nitropyridine,

Compound 26: 2-(1H-indazol-6-yl)amino-6-[(4-pyridyl)methylamino]-3-nitropyridine,

Compound 27: 2-(1H-indazol-5-yl)amino-6-[(2-pyridyl)ethylamino]-3-nitropyridine,

Compound 28: 2-(1H-indazol-6-yl)amino-6-(2-morpholinoethylamino)-3-nitropyridine,

Compound 29: 2-(1H-indazol-5-yl)amino-6-[3-(imidazol-1-yl)propylamino]-3-nitropyridine,

Compound 30: 2-(1H-indazol-5-yl)amino-6-[(2-aminocyclohexyl)amino]-3-nitropyridine,

Compound 31: 2-(1H-indazol-6-yl)amino-6-(methylamino)-3-nitropyridine,

Compound 32: 2-(1H-indazol-5-yl)amino-6-amino-3-nitropyridine,

Compound 33: 2-(1H-indazol-6-yl)amino-6-(isopropylamino)-3-nitropyridine,

Compound 34: 2-(1H-indazol-5-yl)amino-6-(cyclopropylamino)-3-nitropyridine,

Compound 35: 2-(1H-indazol-5-yl)amino-6-[(2-hydroxy-1-hydroxymethyl)-ethylamino]-3-nitropyridine,

Compound 36: 2-(1H-indazol-5-yl)amino-6-[(1-(S)-methyl-2-hydroxyethyl)-amino]-3-nitropyridine,

Compound 37: 2-(1H-indazol-5-yl)amino-6-[(2-morpholino)ethylamino]-3-nitropyridine,

Compound 38: 2-(1H-indazol-6-yl)amino-6-(piperazin-1-yl)-3-nitropyridine,

Compound 39: 2-(1H-indazol-5-yl)amino-6-(4-methylpiperazin-1-yl)-3-nitropyridine,

Compound 40: 2-(1H-indazol-6-yl)amino-6-(4-methylpiperazin-1-yl)-3-nitropyridine,

Compound 41: 2-(1H-indazol-6-yl)amino-6-(4-hydroxypiperidino)-3-nitropyridine,

Compound 42: 2-(1H-indazol-5-yl)amino-6-[(4-methylpiperazin-1-yl)amino]-3-nitropyridine,

Compound 43: 2-(1H-indazol-5-yl)amino-6-[(t-butylamino)amino]-3-nitropyridine,

Compound 44: 2-(1H-indazol-5-yl)amino-6-(3,5-dimethylpiperazin-1-yl)-3-nitropyridine,

Compound 45: 2-(1H-indazol-5-yl)amino-6-[(3-pyridyl)methylamino]-3-nitropyridine,

Compound 46: 2-(1H-indazo -6-yl)amino-6-[(3-pyridyl)methylamino]-3-nitropyridine,

Compound 47: 2-(1H-indazol-5-yl)amino-6-[(4-pyridyl)methylamino]-3-nitropyridine,

Compound 48: 2-(1H-indazol-5-yl)amino-6-[(imidazol-4-yl)ethylamino]-3-nitropyridine,

Compound 49: 2-(1H-indazol-6-yl)amino-6-[(3-imidazol-1-yl)propylamino]-3-nitropyridine,

Compound 50: 2-(1H-indazol-5-yl)amino-6-(4-amino-n-butylamino)-3-nitropyridine, and

Compound 51: 2-(1H-indazol-5-yl)amino-6-[(4-amino)piperidino]-3-nitropyridine.

Further, among the compounds of formula 1 in accordance with the present invention, more preferable compounds are as follows:

Compound 3: 2-(1H-indazol-5-yl)amino-6-(2-isopropylimidazol-1-yl)-3-nitropyridine,

Compound 6: 2-(1H-indazol-5-yl)amino-6-(isopropylamino)-3-nitropyridine,

Compound 8: 2-(1H-indazol-5-yl)amino-6-(isobutylamino)-3-nitropyridine,

Compound 9: 2-(1H-indazol-5-yl)amino-6-(4-hydroxypiperidino)-3-nitropyridine,

Compound 10: 2-(1H-indazol-5-yl)amino-6-(piperazin-1-yl)-3-nitropyridine,

Compound 11: 2-(1H-indazol-5-yl)amino-6-[(N-ethyl-2-hydroxyethyl)amino]-3-nitropyridine,

Compound 12: 2-(1H-indazol-5-yl)amino-6-(t-butylamino)-3-nitropyridine,

Compound 14: 2-(1H-indazol-6-yl)amino-6-[(2-pyridyl)ethylamino]-3-nitropyridine,

Compound 16: 2-(1H-indazol-5-yl)amino-6-[(3-dimethylamino-2,2-dimethyl)propylamino]-3-nitropyridine,

Compound 34: 2-(1H-indazol-5-yl)amino-6-(cyclopropylamino)-3-nitropyridine,

Compound 39: 2-(1H-indazol-5-yl)amino-6-(4-methylpiperazin-1-yl)-3-nitropyridine, and

Compound 50: 2-(1H-indazol-5-yl)amino-6-(4-amino-n-butylamino)-3-nitropyridine.

The 3-nitropyridine derivative which is the compound of formula 1 in accordance with the present invention can be prepared by the method disclosed in Korean Patent Application Nos. 10-1999-0064403 and 10-1999-0053295 or the preparation method of the present invention.

For example, the desired 2,6-substituted-3-nitropyridine compound is prepared by reacting 2,6-dichloro-3-nitropyridine with 5-aminoindazole (or 6-aminoindazole) in an acetonitrile or methanol solvent, in the presence of triethylamine as a base, thereby preparing 6-chloro-2-(1H-indazol-5-yl)amino-3-nitropyridine [or 6-chloro-2-(1H-indazol-6-yl)amino-3-nitropyridine], and reacting 6-chloro-2-(1H-indazol-5-yl)amino-3-nitropyridine [or 6-chloro-2-(1H-indazol-6-yl)amino-3-nitropyridine] with an amine compound capable of introducing a desired substituent at the 6-position of the pyridine ring, in an acetonitrile solvent.

Further, the present invention provides a method for the prevention or treatment of osteoporosis, including administering an effective amount of the compound of formula 1 mentioned in the composition of the present invention to a mammal including a human in need thereof.

Further, the present invention provides use of the compound of formula 1 mentioned in the composition of the present invention, for manufacturing a pharmaceutical preparation for the prevention or treatment of osteoporosis.

The term “osteoporosis” as used herein means the state that minerals and matrices for forming the bone are reduced abnormally in large amounts, even without any defect in the structure of the remaining bone, so that many pores are generated in the bone, making it like a sponge and more likely to fracture. This condition is also referred to as “osteopenia”. In specific embodiments, the 2,6-substituted-3-nitropyridine derivative compound of formula 1 in accordance with the present invention not only promotes the activity of osteoblasts to thereby effectively increase osteogenesis, but also suppresses the formation of osteoclasts to inhibit osteoclastic bone absorption. Thus, the 2,6-substituted-3-nitropyridine derivative compound of the present invention or a pharmaceutically acceptable salt thereof can be beneficially used for the prevention and treatment of osteoporosis.

The composition of the present invention may contain one or more active ingredients which are equivalent or similar in function to the nitropyridine derivative of the present invention, in addition to the 2,6-substituted-3-nitropyridine derivative or a pharmaceutically acceptable salt thereof.

The composition of the present invention which further contains one or more pharmaceutically acceptable carriers in addition to the above-described ingredients may be prepared. The pharmaceutically acceptable carrier may be saline, sterile water, a Ringer's solution, buffered saline, a dextrose solution, a maltodextrin solution, glycerol, ethanol or any combination thereof, and may be, if necessary, further supplemented with other typical additives such as an antioxidant, a buffer and a bacteriostatic agent. In combination with a diluent, a dispersant, a surfactant, a binder and a lubricant, the composition of the present invention may also be formulated into injectable dosage forms, such as an aqueous solution, a suspension and an emulsion, pills, capsules, granules, or tablets. Moreover, depending on the kind of the ingredient or the disease, the formulation may be preferably prepared using an appropriate method known in the art or disclosed in Remington's Pharmaceutical Sciences (latest edition), Mack Publishing Company, Easton, Pa.

The composition of the present invention may be administered orally or parenterally (e.g., intravenously, subcutaneously, intraperitoneally or topically) depending on desired applications. The dosage varies depending on body weight, age, gender, and health state of the patient, diet, administration time period, administration route, excretion rate, and severity of disease. The derivative compound of formula 1 in accordance with the present invention is administered once or several times at a daily dose of approximately 10 to 1,000 mg/kg and preferably at a daily dose of approximately 50 to 500 mg/kg.

For the prevention and treatment of osteoporosis, the composition of the present invention may be used alone or in combination with surgery, hormone therapy, chemical therapy, and use of a biological response modulator.

### Advantageous Effects

2,6-substituted-3-nitropyridine derivatives of the present invention inhibit osteoclastic bone absorption and promotes osteoblast osteogenesis and therefore can be used as an agent for the prevention and treatment of osteoporosis.

### Mode for Invention

A better understanding of the present invention may be obtained through the following preferable Preparation Examples and Examples, which are set forth to illustrate, but are not to be construed as the limit of the present invention.

Unless otherwise specified, reagents and solvents referred hereinafter were purchased from Aldrich or Cambridge Isotope Laboratories, and ¹H-NMR data were measured by a JNM-LA400 spectrometer (manufactured by JEOL) and Mass data were measured by a 1100MSD spectrometer (manufactured by Hewlett Packard).

### PREPARATION EXAMPLE 1

### Preparation of 6-chloro-2-(1H-indazol-5-yl)amino-3-nitropyridine compound

To 150 ml of methanol were added 5 g (25.9 mmol) of 2,6-dichloronitropyridine and 4 ml (28.5 mmol) of triethylamine and 3.6 g (27.2 mmol) of 5-aminoindazole was then slowly added thereto at a temperature of 0 to 5°, followed by reaction at room temperature (20 to 30°) for about 8 hours. After the reaction was completed, the reactant was filtered, washed with 30 ml of methanol and then dried under vacuum at about 40° to afford 6.4 g (yield: 85%) of the desired compound.

Mass (M+): 290.1

¹H-NMR (DMSO-d₆): 6.94(d, 1H), 7.44(d, 1H), 7.56(d, 1H), 7.91(s, 1H), 8.08(s, 1H), 8.53(d, 1H), 10.20(s, 1H), 13.12(brs, 1H).

### EXAMPLE 1

### Preparation of 2-(1H-indazol-5-yl)amino-6-(2-methyl-4,5-dihydro-imidazol-1-yl)amino-3-nitropyridine (Compound 1)

To 30 ml of acetonitrile were sequentially added 300 mg (1.04 mmol) of the 6-chloro-2-(1H-indazol-5-yl)amino-3-nitropyridine compound obtained in Preparation Example 1, 0.29 ml (2.07 mmol) of triethylamine and 176 mg (2.07mmol) of 2-methyl-2-imidazoline, followed by reaction at a temperature of 65 to 70° for 8 hours. After the reaction was completed, the reaction liquid was cooled to room temperature, and the resulting solid was filtered, washed with 10 ml of methanol and then dried under vacuum at about 40° to afford 251 mg (yield: 72%) of the desired compound.

Mass (M+): 338.0

¹H-NMR(DMSO-d₆) (ppm) 1.75(s, 3H), 3.64(t, 2H), 3.80(t, 2H), 6.34(d, 1H), 7.35(dd, 1H), 7.52(d, 1H), 7.63(d, 1H), 8.04(s, 1H), 8.35(d, 1H), 10.24(brs, 1H), 13.09(s, 1H).

### EXAMPLES 2 TO 4

In the same manner as in Example 1 and using amine compounds described in the following Table 1 in place of “2-methyl-2-imidazoline” and using “acetonitrile” as the reaction solvent, the following desired compounds can be synthesized by adjusting equivalents of the to-be-substituted amines depending on the difference in reactivity among the to-be-substituted amine compounds during carrying out the reaction, or by adjusting the reaction temperature, or by adjusting use of a tertiary organic base such as triethylamine upon carrying out the reaction. Taking into consideration these various factors, the desired compounds described in the following Table 1 were obtained.

The following Table 1 shows the name of Compound 1 to Compound 4 prepared in Examples 2 to 4, the name and equivalents of amine compounds used in the reaction, use/nonuse and equivalents of triethylamine in the reaction, the reaction temperature, the reaction solvent, yield, Mass analysis results and NMR analysis results.

TABLE 1

| | Amine compound | Use/nonuse of | | | | Reaction | | |
|---|---|---|---|---|---|---|---|---|
| Example No. | used | Et₃N | Name of | NMR | | temperature | Yield | |
| (Compound No.) | (equivalents *) | (equivalents *) | compound | (DMSO-d₆) | Solvent | ° C. | (%) | M(+) |
| 2 | 2-methyl- | ∘ | 2-(1H-indazol- | 2.06(s, 3H), 6.85(d, | CH₃C | 60~70 | 69 | 336.0 |
| (2) | imidazole | (10 equivalents) | 5-yl)amino-6- | 1H), 7.09(d, 1H), | | | | |
| | (10 equivalents) | | (2-methyl- | 7.43(dd, 1H), | | | | |
| | | | imidazol-1-yl)- | 7.55(d, 1H), 7.62(d, | | | | |
| | | | 3-nitropyridine | 1H), 7.83(d, 1H), | | | | |
| | | | | 8.06(s, 1H), 8.66(d, | | | | |
| | | | | 1H), 10.23(s, 1H), | | | | |
| | | | | 13.12(s, 1H). | | | | |
| 3 | 2-isopropyl- | ∘ | 2-(1H-indazol- | 0.67(d, 6H), | CH₃C | 60~70 | 35 | 364.1 |
| (3) | imidazole | (5 equivalents) | 5-yl)amino-6- | 3.17(m, 1H), 6.86(s, | | | | |
| | (5 equivalents) | | (2-isopropyl- | 1H), 7.04(d, 1H), | | | | |
| | | | imidazol-1-yl)- | 7.35(d, 1H), | | | | |
| | | | 3-nitropyridine | 7.59(m, 2H), 7.77(s, | | | | |
| | | | | 1H), 8.08(s, 1H), | | | | |
| | | | | 8.64(d, 1H), | | | | |
| | | | | 10.21(s, 1H). | | | | |
| 4 | 5-methyl- | ∘ | 2-(1H-indazol- | 2.09(s, 3H), 7.21(s, | CH₃C | 60~70 | 68 | 336.0 |
| (4) | imidazole | (10 equivalents) | 5-yl)amino-6- | 1H), 7.45(s, 1H), | | | | |
| | (10 equivalents) | | (5-methyl- | 7.60(m, 2H), 8.05(s, | | | | |
| | | | imidazol-1-yl)- | 1H), 8.08(s, 1H), | | | | |
| | | | 3-nitropyridine | 8.25(s, 1H), | | | | |
| | | | | 8.65(dd, 1H), | | | | |
| | | | | 10.27(s, 1H), | | | | |
| | | | | 13.12(s, 1H). | | | | |

In the above table, * means equivalents used based on the starting material, 6-chloro-2-(1H-indazol-5-yl)amino-3-nitropyridine obtained in Preparation Example 1, “0” means additional use of triethylamine, and “X” means no additional use of triethylamine.

### EXPERIMENTAL EXAMPLE 1

### Osteoclastogenesis Inhibitory Effects of Compounds Via Co-Culture System

Osteoclastogenesis inhibitory effects of the compounds of the present invention were evaluated via a co-culture system (Reference: Endocrinology 137(1996), 2187 to 2190, E. Jimi et al.). A specific experimental method is as follows.

1) Preparation of Bone Marrow Cells and Osteoblasts

Femur and tibia were aseptically dissected from 6 to 8-week-old male ddY mice to harvest bone marrow cells by a conventional method using a syringe. In brief, tissues were removed from the dissected bone, the bone ends were cut off with scissors, and the bone marrow was isolated by pushing a medium-containing syringe (23G) against the one end of the cut bone. The isolated bone marrow was subjected to repeated piston movement of a syringe such that single cells were obtained (Reference: Endocrinology 123(1988), 2600 to 2602, Takahashi et al.). After removal of red blood cells within the bone marrow, the bone marrow cells recovered by centrifugation were placed in an α-MEM supplemented with 10% fetal bovine serum (FBS), followed by counting of nucleated cells and then were immediately used for a co-culture system.

For the preparation of osteoblasts (Calvarial cells), the cranial bone were aseptically dissected from 1 to 2-day-old neonatal ICR mice and subjected to continuous reaction with a 0.2% collagenase solution to separate osteoblasts. The cell-suspended supernatant was centrifuged to recover osteoblasts which were grown to full confluence in an α-MEM supplemented with 10% FBS and then diluted to a desired cell density for use in the experiment.

2) Osteoclastogenesis Inhibition Experiment Via Co-Culture System

As the medium used for a co-culture system, a differentiation medium with the addition of differentiation factors 1α,25-dihydroxyvitamin D3 (10⁻⁸M) and dexamethasone (10⁻⁸M) to α-MEM supplemented with 10% FBS was used for the induction of osteoclastogenesis. First, the compounds of the present invention dissolved in dimethyl sulfoxide (DMSO) at a concentration of 1 mM were diluted to 2 μM using the above-mentioned differentiation medium. As a vehicle control group, 0.2% (v/v) DMSO was added to the medium. 100 μL/well of each medium was added to 96-well plates. In addition, the above prepared bone marrow cells and osteoblasts were plated onto 96-well plates at a density of 1×10⁵ cells/50 μL/well and 3×10³ cells/50 μL/well, respectively. The total volume/well was 200 μL and the final compound concentration was 1 μM. The control group was 0.1% (v/v) DMSO. The cells were cultured with exchange of the culture media with fresh media containing differentiation factors and test materials (materials containing the compounds of the present invention) at an interval of 2 to 3 days.

7 days after culturing of cells, the formation of multinucleated osteoclasts was confirmed by microscopic examination, the medium was removed from the wells and then the cells were fixed in a 10% phosphate-buffered formalin solution. The degree of formation of mature osteoclasts was measured taking advantage of the characteristics of osteoclasts showing a positive reaction to a tartrate-resistant acid phosphatase (TRAP) staining solution. The TRAP staining solution was prepared in a manner such that naphthol AS-MS phosphate as a substrate and a coloring agent (Fast Red Violet LB salt) were dissolved in N,N-dimethylformamide, and a 0.1N NaHCO₃ buffer solution containing 50 mM of tartaric acid was added thereto. Among the TRAP-positive cells under a light microscope, multinucleated osteoclasts having 6 to 7 nuclei were regarded as mature osteoclasts.

The degree of inhibition of osteoclastogenesis was calculated according to the following equation 1. The results are summarized in Table 2 below (Experiments were carried out for 4 wells/experimental group (n=4), and the results are given in terms of mean±standard deviation)

Inhibition of osteoclastogenesis(%)=(1−numbers of osteoclasts observed in experimental group/numbers of osteoclasts observed in vehicle control group)×100(%) [Equation 1]

TABLE 2

| | Osteoclastogenesis |
|---|---|
| | inhibition (%) |
| | 1 μM |
| Compound 2 | 70 |
| Compound 3 | 95 |
| Compound 4 | 67 |
| Compound 5 | 89 |
| Compound 6 | 100 |
| Compound 7 | 72 |
| Compound 8 | 100 |
| Compound 9 | 100 |
| Compound 10 | 99 |
| Compound 11 | 98 |
| Compound 12 | 99 |
| Compound 13 | 88 |
| Compound 14 | 98 |
| Compound 15 | 98 |
| Compound 16 | 89 |
| Compound 17 | 92 |
| Compound 19 | 90 |
| Compound 20 | 90 |
| Compound 22 | 100 |
| Compound 23 | 92 |
| Compound 24 | 86 |
| Compound 25 | 81 |
| Compound 26 | 78 |
| Compound 27 | 90 |
| Compound 28 | 74 |
| Compound 29 | 73 |
| Compound 30 | 100 |
| Compound 32 | 100 |
| Compound 34 | 100 |
| Compound 36 | 72 |
| Compound 38 | 90 |
| Compound 39 | 80 |
| Compound 44 | 90 |
| Compound 47 | 71 |
| Compound 50 | 100 |

As shown in Table 2 above, most of the compounds of the present invention were observed to exhibit approximately 95% inhibition of osteoclastogenesis. Therefore, it was demonstrated that the compounds of the present invention can be used as a superior preventive and therapeutic agent against osteoporosis.

### EXPERIMENTAL EXAMPLE 2

### Evaluation of Alkaline Phosphatase (ALP) Activity

Differentiation and activity of osteoblasts were indirectly evaluated by measuring an ALP activity having a close relationship with osteogenesis.

Osteoblasts (Calvarial cells) prepared in Experimental Example 1 and MC3T3-E1 cells (available from RIKEN Cell Bank, Japan) were collected in α-MEM supplemented with 10% FBS, followed by cell counting. The cells were dispensed into 24-well cultureware at a density of 2×10⁴ cells/well. After culturing of the cells for 24 hours, the culture media were discarded and replaced with fresh media in which test compounds were diluted to a concentration of 1 μM (1 mL/well). In addition, the vehicle control group containing 0.1% DMSO was also treated. Under the conditions where the compounds were treated, the cells were cultured in a 5% CO₂ inhibitor at 37° for 3 days. When the experiment was terminated, the supernatant was removed and the cells were washed three times with cold phosphate buffer at 4°. 0.2% Triton X-100 was added to the washed cells which were then subjected to three cycles of freezing at −70° and thawing at room temperature for the complete lysis of cells. The cell extracts were pooled and centrifuged to collect the cell supernatant which was used for the measurement of ALP activity and proteins. The protein concentration was measured using a BCA assay kit (manufactured by Sigma-Aldrich). For the measurement of ALP activity, p-nitrophenylphosphate was added to the cell supernatant which was then incubated at 37° for 30 minutes, and the reaction was terminated with the addition of 50 μL of 0.2N sodium hydroxide. The standard curve was plotted at the absorbance of 405 nm using p-nitrophenol as a standard material and then the absorbance of test materials thus reacted was measured to determine the production amount of p-nitrophenol.

The ALP activity was calculated by dividing the amount of p-nitrophenol produced from each test material by the protein amount and the reaction time. Therefore, the unit of ALP activity was given in terms of p-nitrophenol/μg protein/min. The results are given in Table 3 where the ALP activity unit of each test material was given in terms of % change through the comparison between the individual test materials and the vehicle control group.

TABLE 3

| | ALP activity | |
|---|---|---|
| | (1 μM, % of Control) | |
| | Calvarial | MC3T3-E1 |
| | cell | cell |
| Compound 1 | 158 | 168 |
| Compound 3 | 148 | 172 |
| Compound 5 | 193 | 206 |
| Compound 6 | 188 | 207 |
| Compound 7 | 221 | 155 |
| Compound 8 | 157 | 240 |
| Compound 9 | 166 | 112 |
| Compound 10 | 189 | 124 |
| Compound 11 | 143 | 140 |
| Compound 12 | 152 | 136 |
| Compound 14 | 111 | 121 |
| Compound 16 | 123 | 144 |
| Compound 17 | 139 | 188 |
| Compound 19 | 132 | — |
| Compound 20 | 119 | — |
| Compound 21 | 115 | — |
| Compound 22 | 119 | — |
| Compound 23 | 139 | — |
| Compound 24 | 107 | — |
| Compound 25 | 128 | — |
| Compound 26 | 113 | — |
| Compound 33 | 122 | — |
| Compound 34 | 154 | — |
| Compound 35 | — | 123 |
| Compound 37 | 126 | 141 |
| Compound 39 | 159 | — |
| Compound 49 | 129 | — |
| Control | 100 | 100 |

In Table 3 above, “—” means “not determined”.

As shown in Table 3, it was demonstrated that the compounds of the present invention exhibit excellent ALP activity on both Calvarial cells and MC3T3-E1 cells.

### EXPERIMENTAL EXAMPLE 3

### Cytotoxicity Test

Cytotoxicity of the compounds of the present invention was evaluated by carrying out the experiment described below.

Drugs were diluted to a concentration of 2 μM in α-MEM culture media supplemented with 10% FBS. The vehicle control group was established to contain 0.2% DMSO. 100 μL/well of the diluted drugs were dispensed into 96-well plates to which osteoblasts (calvarial cells) prepared in Experimental Example 1 were then added at a density of 1.0×10⁴ cells/100 μL/well. Here, the final compound concentration in the cell culture was 1 μM, and the vehicle control group contained 0.1% DMSO. The cells were cultured in a 5% CO₂ inhibitorr at 37° for 72 hours. 25 μL of 3-(4,5-dimethyl-2-thiazolyl)-2,5-diphenyl-2H-tetrazolium bromide (MTT) dissolved in PBS (2 mg/mL) was added to each cell culture 4 hours before the end of culture. After completion of the reaction, the plates were centrifuged, the media were discarded, and 100 μL of formazan was added and dissolved in dimethyl sulfoxide (DMSO). Finally, the absorbance of the developed plates was measured at 540 nm. The cell viability was expressed as % concentration in comparison with the vehicle control group. The results are given in Table 4.

TABLE 4

| | Cell viability (%) |
|---|---|
| | Calvarial cell |
| Compound 1 | 89 |
| Compound 2 | 104 |
| Compound 3 | 118 |
| Compound 4 | 101 |
| Compound 5 | 112 |
| Compound 6 | 97 |
| Compound 7 | 103 |
| Compound 8 | 96 |
| Compound 9 | 105 |
| Compound 10 | 95 |
| Compound 11 | 105 |
| Compound 12 | 109 |
| Compound 13 | 94 |
| Compound 14 | 91 |
| Compound 15 | 90 |
| Compound 16 | 104 |
| Compound 17 | 89 |
| Compound 18 | 98 |
| Compound 19 | 100 |
| Compound 21 | 96 |
| Compound 22 | 99 |
| Compound 23 | 98 |
| Compound 24 | 110 |
| Compound 25 | 106 |
| Compound 26 | 106 |
| Compound 27 | 97 |
| Compound 28 | 104 |
| Compound 29 | 117 |
| Compound 30 | 114 |
| Compound 31 | 97 |
| Compound 32 | 99 |
| Compound 33 | 97 |
| Compound 34 | 93 |
| Compound 35 | 104 |
| Compound 36 | 103 |
| Compound 37 | 94 |
| Compound 38 | 91 |
| Compound 39 | 97 |
| Compound 40 | 95 |
| Compound 41 | 98 |
| Compound 42 | 106 |
| Compound 43 | 100 |
| Compound 44 | 96 |
| Compound 45 | 103 |
| Compound 46 | 99 |
| Compound 47 | 102 |
| Compound 48 | 96 |
| Compound 49 | 95 |
| Compound 50 | 95 |
| Compound 51 | 73 |

As shown in Table 4, it was demonstrated that the compounds of the present invention show substantially no cytotoxicity.

1. A 2,6-substituted-3-nitropyridine derivative compound that is selected from among:
2-(1H-indazol-5-yl)amino-6-(2-methyl-4,5-dihydroimidazol-1-yl)amino-3-nitropyridine;
2-(1H-indazol-5-yl)amino-6-(2-methylimidazol-1-yl)-3-nitropyridine;
2-(1H-indazol-5-yl)amino-6-(2-isopropylimidazol-1-yl)-3-nitropyridine; and
2-(1H-indazol-5-yl)amino-6-(5-methylimidazol-1-yl)-3-nitropyridine;
or a pharmaceutically acceptable salt thereof.

2. A method for preparing a 2,6-substituted-3-nitropyridine derivative compound, comprising:
a) reacting 2,6-dichloro-3-nitropyridine with 5-aminoindazole in the presence of a base to prepare a 2-(1H-indazol-5-yl)amino-6-chloro-3-nitropyridine; and
b) reacting the 2-(1H-indazol-5-yl)amino-6-chloro-3-nitropyridine prepared in Step a) with an amine compound selected from 2-methyl-2-imidazoline, 2-methylimidazole, 2-isopropylimidazole and 5-methylimidazole to prepare the 2,6-substituted-3-nitropyridine derivative compound of claim 1 in which the amine compound is introduced at the 6-position of the pyridine ring.

3. The method of claim 2, wherein the base of Step a) is at least one selected from among triethylamine, N,N-diisopropylethylamine, N-methylmorpholine, N-methylpiperidine, 4-dimethylaminopyridine, N,N-dimethylaniline, 2,6-lutidine and pyridine.

4. A composition for the prevention or treatment of osteoporosis, comprising:
an effective amount of a 3-nitropyridine derivative compound of formula 1: 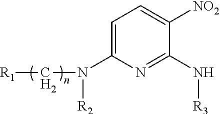
wherein:
R₁ is H, hydroxy, amino, C₃-C₆ linear or branched aminoalkyl, C₂-C₆ dialkylamino, C₂-C₆ linear or branched hydroxyalkyl, C₃-C₆ linear or branched dihydroxyalkyl, C₁-C₃ alkoxy-substituted C₁-C₆ linear or branched alkyl, or a saturated or unsaturated 5- or 6-membered heterocyclic ring that contains 1 to 3 hetero atoms selected from among N, O and S and that is unsubstituted or substituted by C₁-C₃ alkyl, and R₁ optionally contains an asymmetric carbon atom;
R₂ is H, C₁-C₄ linear or branched alkyl, or unsubstituted or substituted C₃-C₆ cyclicalkyl, or
R₁ and R₂ taken together form a saturated or unsaturated 5 or 6-membered heterocyclic ring, wherein the heterocyclic ring contains 1 to 3 hetero atoms selected from among N, O and S and is unsubstituted or substituted by one or more groups selected from among C₁-C₅ linear or branched alkyl, amino, C₂-C₅ linear or branched hydroxyalkyl and hydroxy;
R₃ is indazol-5-yl or indazol-6-yl, and
n is an integer of 0 to 4;
or a pharmaceutically acceptable salt thereof.

5. The composition of claim 4, wherein:
R₁ is H, an amine group, hydroxy, methoxy, pyridinyl, imidazolyl, 1,3-dioxalane, morpholine, unsubstituted or substituted piperazine, N-methylpiperazine, 3-dimethylamino-2,2-dimethylpropyl or NR₄R₅, wherein R₄ and R₅ each independently is hydrogen, methyl, t-butyl, morpholinyl or N-methylpiperazine; and
R₂ is H, methyl, ethyl, isopropyl, isobutyl, cyclic propyl, 2-amino cyclic hexyl or t-butyl; or
R₁ and R₂ taken together form 4-hydroxypiperidin-1-yl, 4-aminopiperidin-1-yl, piperazin-1-yl, 4-methylpiperazin-1-yl, 3,5-dimethylpiperazin-1-yl, 2,2,6,6-tetramethyl-piperazin-1-yl, 4-hydroxyethylpiperazin-1-yl, methylimidazoline, ethylimidazolyl or isopropylimidazolyl;
R₃ is indazol-5-yl or indazol-6-yl; and
n is an integer of 0 to 4.

6. The composition of claim 5, wherein the compound of formula 1 is at least one selected from among:
2-(1H-indazol-5-yl)amino-6-(2-methyl-4,5-dihydroimidazol-1-yl)amino-3-nitropyridine;
2-(1H-indazol-5-yl)amino-6-(2-methylimidazol-1-yl)-3-nitropyridine;
2-(1H-indazol-5-yl)amino-6-(2-isopropylimidazol-1-yl)-3-nitropyridine;
2-(1H-indazol-5-yl)amino-6-(5-methylimidazol-1-yl)-3-nitropyridine;
2-(1H-indazol-5-yl)amino-6-(methylamino)-3-nitropyridine;
2-(1H-indazol-5-yl)amino-6-(isopropylamino)-3-nitropyridine;
2-(1H-indazol-5-yl)amino-6-[(N-methyl-2-hydroxyethyl)amino]-3-nitropyridine;
2-(1H-indazol-5-yl)amino-6-(isobutylamino)-3-nitropyridine;
2-(1H-indazol-5-yl)amino-6-(4-hydroxypiperidino)-3-nitropyridine;
2-(1H-indazol-5-yl)amino-6-(piperazin-1-yl)-3-nitropyridine;
2-(1H-indazol-5-yl)amino-6-[(N-ethyl-2-hydroxyethyl)amino]-3-nitropyridine;
2-(1H-indazol-5-yl)amino-6-(t-butylamino)-3-nitropyridine;
2-(1H-indazol-5-yl)amino-6-(2,2,6,6-tetramethylpiperazin-4-yl)-3-nitropyridine;
2-(1H-indazol-6-yl)amino-6-[(2-pyridyl)ethylamino]-3-nitropyridine;
2-(1H-indazol-5-yl)amino-6-[4-(2-hydroxyethyl)piperazin-1-yl]-3-nitropyridine;
2-(1H-indazol-5-yl)amino-6-[(3-dimethylamino-2,2-dimethyl)propylamino]-3-nitropyridine;
2-(1H-indazol-5-yl)amino-6-[(N-[1,3]-dioxolan-2-ylmethyl)-methylamino]-3-nitropyridine;
2-(1H-indazol-6-yl)amino-6-[(1-(S)-methyl-2-hydroxyethyl)amino]-3-nitropyridine;
2-(1H-indazol-6-yl)amino-6-[4-(2-hydroxyethyl)piperazin-1-yl]-3-nitropyridine;
2-(1H-indazol-6-yl)amino-6-[2-(N,N-dimethylamino)ethylamino]-3-nitropyridine;
2-(1H-indazol-6-yl)amino-6-[(2-pyridyl)methylamino]-3-nitropyridine;
2-(1H-indazol-5-yl)amino-6-[(1-methyl-2-methoxy)ethylamino]-3-nitropyridine;
2-(1H-indazol-6-yl)amino-6-[(4-methylpiperazin-1-yl)amino]-3-nitropyridine;
2-(1H-indazol-6-yl)amino-6-[(4-morpholin-1-yl)amino]-3-nitropyridine;
2-(1H-indazol-5-yl)amino-6-(N,N-dimethylamino)-3-nitropyridine;
2-(1H-indazol-6-yl)amino-6-[(4-pyridyl)methylamino]-3-nitropyridine;
2-(1H-indazol-5-yl)amino-6-[(2-pyridyl)ethylamino]-3-nitropyridine;
2-(1H-indazol-6-yl)amino-6-(2-morpholinoethylamino)-3-nitropyridine;
2-(1H-indazol-5-yl)amino-6-[3-(imidazol-1-yl)propylamino]-3-nitropyridine;
2-(1H-indazol-5-yl)amino-6-[(2-aminocyclohexyl)amino]-3-nitropyridine;
2-(1H-indazol-6-yl)amino-6-(methylamino)-3-nitropyridine;
2-(1H-indazol-5-yl)amino-6-amino-3-nitropyridine;
2-(1H-indazol-6-yl)amino-6-(isopropylamino)-3-nitropyridine;
2-(1H-indazol-5-yl)amino-6-(cyclopropylamino)-3-nitropyridine;
2-(1H-indazol-5-yl)amino-6-[(2-hydroxy-1-hydroxymethyl)ethylamino]-3-nitropyridine;
2-(1H-indazol-5-yl)amino-6-[(1-(S)-methyl-2-hydroxyethyl)amino]-3-nitropyridine;
2-(1H-indazol-5-yl)amino-6-[(2-morpholino)ethylamino]-3-nitropyridine;
2-(1H-indazol-6-yl)amino-6-(piperazin-1-yl)-3-nitropyridine;
2-(1H-indazol-5-yl)amino-6-(4-methylpiperazin-1-yl)-3-nitropyridine;
2-(1H-indazol-6-yl)amino-6-(4-methylpiperazin-1-yl)-3-nitropyridine;
2-(1H-indazol-6-yl)amino-6-(4-hydroxypiperidino)-3-nitropyridine;
2-(1H-indazol-5-yl)amino-6-[(4-methylpiperazin-1-yl)amino]-3-nitropyridine;
2-(1H-indazol-5-yl)amino-6-[(t-butylamino)amino]-3-nitropyridine;
2-(1H-indazol-5-yl)amino-6-(3,5-dimethylpiperazin-1-yl)-3-nitropyridine;
2-(1H-indazol-5-yl)amino-6-[(3-pyridyl)methylamino]-3-nitropyridine;
2-(1H-indazol-6-yl)amino-6-[(3-pyridyl)methylamino]-3-nitropyridine;
2-(1H-indazol-5-yl)amino-6-[(4-pyridyl)methylamino]-3-nitropyridine;
2-(1H-indazol-5-yl)amino-6-[(imidazol-4-yl)ethylamino]-3-nitropyridine;
2-(1H-indazol-6-yl)amino-6-[(3-imidazol-1-yl)propylamino]-3-nitropyridine;
2-(1H-indazol-5-yl)amino-6-(4-amino-n-butylamino)-3-nitropyridine;
2-(1H-indazol-5-yl)amino-6-[(4-amino)piperidino]-3-nitropyridine;
and pharmaceutically acceptable salts thereof.

7. The composition of claim 6, wherein the compound of formula 1 is selected from among:
2-(1H-indazol-5-yl)amino-6-(2-isopropylimidazol-1-yl)-3-nitropyridine;
2-(1H-indazol-5-yl)amino-6-(isopropylamino)-3-nitropyridine;
2-(1H-indazol-5-yl)amino-6-(isobutylamino)-3-nitropyridine;
2-(1H-indazol-5-yl)amino-6-(4-hydroxypiperidino)-3-nitropyridine;
2-(1H-indazol-5-yl)amino-6-(piperazin-1-yl)-3-nitropyridine;
2-(1H-indazol-5-yl)amino-6-[(N-ethyl-2-hydroxyethyl)amino]-3-nitropyridine;
2-(1H-indazol-5-yl)amino-6-(t-butylamino)-3-nitropyridine;
2-(1H-indazol-6-yl)amino-6-[(2-pyridyl)ethylamino]-3-nitropyridine;
2-(1H-indazol-5-yl)amino-6-[(3-dimethylamino-2,2-dimethyl)propylamino]-3-nitropyridine;
2-(1H-indazol-5-yl)amino-6-(cyclopropylamino)-3-nitropyridine;
2-(1H-indazol-5-yl)amino-6-(4-methylpiperazin-1-yl)-3-nitropyridine;
2-(1H-indazol-5-yl)amino-6-(4-amino-n-butylamino)-3-nitropyridine;
and pharmaceutically acceptable salts thereof.

8. The composition of claim 4, wherein the pharmaceutically acceptable salt is hydrochloride or methanesulfonate.

9.-13. (canceled)

14. A method for the prevention or treatment of osteoporosis, comprising:
administering to a mammal an effective amount of a 3-nitropyridine derivative compound of formula 1: 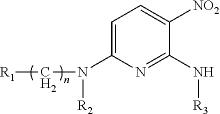
wherein:
R₁ is H, hydroxy, amino, C₃-C₆ linear or branched aminoalkyl, C₂-C₆ dialkylamino, C₂-C₆ linear or branched hydroxyalkyl, C₃-C₆ linear or branched dihydroxyalkyl, C₁-C₃ alkoxy-substituted C₁-C₆ linear or branched alkyl, or a saturated or unsaturated 5- or 6-membered heterocyclic ring that contains 1 to 3 hetero atoms selected from among N, O and S and that is unsubstituted or substituted by C₁-C₃ alkyl, and R₁ optionally contains an asymmetric carbon atom; and
R₂ is H, C₁-C₄ linear or branched alkyl, or unsubstituted or substituted C₃-C₆ cyclicalkyl, or
R₁ and R₂ taken together form a saturated or unsaturated 5 or 6-membered heterocyclic ring, wherein the heterocyclic ring contains 1 to 3 hetero atoms selected from among N, O and S and is unsubstituted or substituted by one or more groups selected from among C₁-C₅ linear or branched alkyl, amino, C₂-C₅ linear or branched hydroxyalkyl and hydroxy,
R₃ is indazol-5-yl or indazol-6-yl, and
n is an integer of 0 to 4;
or a pharmaceutically acceptable salt thereof,
whereby osteoporosis is prevented or treated.

15. The method of claim 14, wherein:
R₁ is H, an amine group, hydroxy, methoxy, pyridinyl, imidazolyl, 1,3-dioxalane, morpholine, unsubstituted or substituted piperazine, N-methylpiperazine, 3-dimethylamino-2,2-dimethylpropyl or NR₄R₅, wherein R₄ and R₅ each independently is hydrogen, methyl, t-butyl, morpholinyl or N-methylpiperazine; and
R₂ is H, methyl, ethyl, isopropyl, isobutyl, cyclic propyl, 2-amino cyclic hexyl or t-butyl; or
R₁ and R₂ taken together form 4-hydroxypiperidin-1-yl, 4-aminopiperidin-1-yl, piperazin-1-yl, 4-methylpiperazin-1-yl, 3,5-dimethylpiperazin-1-yl, 2,2,6,6-tetramethylpiperazin-1-yl, 4-hydroxyethylpiperazin-1-yl, methylimidazoline, ethylimidazolyl or isopropylimidazolyl;
R₃ is indazol-5-yl or indazol-6-yl; and
n is an integer of 0 to 4.

16. The method of claim 15, wherein the compound of formula 1 is at least one selected from among:
2-(1H-indazol-5-yl)amino-6-(2-methyl-4,5-dihydroimidazol-1-yl)amino-3-nitropyridine;
2-(1H-indazol-5-yl)amino-6-(2-methylimidazol-1-yl)-3-nitropyridine;
2-(1H-indazol-5-yl)amino-6-(2-isopropylimidazol-1-yl)-3-nitropyridine;
2-(1H-indazol-5-yl)amino-6-(5-methylimidazol-1-yl)-3-nitropyridine;
2-(1H-indazol-5-yl)amino-6-(methylamino)-3-nitropyridine;
2-(1H-indazol-5-yl)amino-6-(isopropylamino)-3-nitropyridine;
2-(1H-indazol-5-yl)amino-6-[(N-methyl-2-hydroxyethyl)amino]-3-nitropyridine;
2-(1H-indazol-5-yl)amino-6-(isobutylamino)-3-nitropyridine;
2-(1H-indazol-5-yl)amino-6-(4-hydroxypiperidino)-3-nitropyridine;
2-(1H-indazol-5-yl)amino-6-(piperazin-1-yl)-3-nitropyridine;
2-(1H-indazol-5-yl)amino-6-[(N-ethyl-2-hydroxyethyl)amino]-3-nitropyridine;
2-(1H-indazol-5-yl)amino-6-(t-butylamino)-3-nitropyridine;
2-(1H-indazol-5-yl)amino-6-(2,2,6,6-tetramethylpiperazin-4-yl)-3-nitropyridine;
2-(1H-indazol-6-yl)amino-6-[(2-pyridyl)ethylamino]-3-nitropyridine;
2-(1H-indazol-5-yl)amino-6-[4-(2-hydroxyethyl)piperazin-1-yl]-3-nitropyridine;
2-(1H-indazol-5-yl)amino-6-[(3-dimethylamino-2,2-dimethyl)propylamino]-3-nitropyridine;
2-(1H-indazol-5-yl)amino-6-[(N-[1,3]-dioxolan-2-ylmethyl)-methylamino]-3-nitropyridine;
2-(1H-indazol-6-yl)amino-6-[(1-(S)-methyl-2-hydroxyethyl)amino]-3-nitropyridine;
2-(1H-indazol-6-yl)amino-6-[4-(2-hydroxyethyl)piperazin-1-yl]-3-nitropyridine;
2-(1H-indazol-6-yl)amino-6-[2-(N,N-dimethylamino)ethylamino]-3-nitropyridine;
2-(1H-indazol-6-yl)amino-6-[(2-pyridyl)methylamino]-3-nitropyridine;
2-(1H-indazol-5-yl)amino-6-[(1-methyl-2-methoxy)ethylamino]-3-nitropyridine;
2-(1H-indazol-6-yl)amino-6-[(4-methylpiperazin-1-yl)amino]-3-nitropyridine;
2-(1H-indazol-6-yl)amino-6-[(4-morpholin-1-yl)amino]-3-nitropyridine;
2-(1H-indazol-5-yl)amino-6-(N,N-dimethylamino)-3-nitropyridine;
2-(1H-indazol-6-yl)amino-6-[(4-pyridyl)methylamino]-3-nitropyridine;
2-(1H-indazol-5-yl)amino-6-[(2-pyridyl)ethylamino]-3-nitropyridine;
2-(1H-indazol-6-yl)amino-6-(2-morpholinoethylamino)-3-nitropyridine;
2-(1H-indazol-5-yl)amino-6-[3-(imidazol-1-yl)propylamino]-3-nitropyridine;
2-(1H-indazol-5-yl)amino-6-[(2-aminocyclohexyl)amino]-3-nitropyridine;
2-(1H-indazol-6-yl)amino-6-(methylamino)-3-nitropyridine;
2-(1H-indazol-5-yl)amino-6-amino-3-nitropyridine;
2-(1H-indazol-6-yl)amino-6-(isopropylamino)-3-nitropyridine;
2-(1H-indazol-5-yl)amino-6-(cyclopropylamino)-3-nitropyridine;
2-(1H-indazol-5-yl)amino-6-[(2-hydroxy-1-hydroxymethyl)ethylamino]-3-nitropyridine;
2-(1H-indazol-5-yl)amino-6-[(1-(S)-methyl-2-hydroxyethyl)amino]-3-nitropyridine;
2-(1H-indazol-5-yl)amino-6-[(2-morpholino)ethylamino]-3-nitropyridine;
2-(1H-indazol-6-yl)amino-6-(piperazin-1-yl)-3-nitropyridine;
2-(1H-indazol-5-yl)amino-6-(4-methylpiperazin-1-yl)-3-nitropyridine;
2-(1H-indazol-6-yl)amino-6-(4-methylpiperazin-1-yl)-3-nitropyridine;
2-(1H-indazol-6-yl)amino-6-(4-hydroxypiperidino)-3-nitropyridine;
2-(1H-indazol-5-yl)amino-6-[(4-methylpiperazin-1-yl)amino]-3-nitropyridine;
2-(1H-indazol-5-yl)amino-6-[(t-butylamino)amino]-3-nitropyridine;
2-(1H-indazol-5-yl)amino-6-(3,5-dimethylpiperazin-1-yl)-3-nitropyridine;
2-(1H-indazol-5-yl)amino-6-[(3-pyridyl)methylamino]-3-nitropyridine;
2-(1H-indazol-6-yl)amino-6-[(3-pyridyl)methylamino]-3-nitropyridine;
2-(1H-indazol-5-yl)amino-6-[(4-pyridyl)methylamino]-3-nitropyridine;
2-(1H-indazol-5-yl)amino-6-[(imidazol-4-yl)ethylamino]-3-nitropyridine;
2-(1H-indazol-6-yl)amino-6-[(3-imidazol-1-yl)propylamino]-3-nitropyridine;
2-(1H-indazol-5-yl)amino-6-(4-amino-n-butylamino)-3-nitropyridine;
2-(1H-indazol-5-yl)amino-6-[(4-amino)piperidino]-3-nitropyridine;
and pharmaceutically acceptable salts thereof.

17. The method of claim 16, wherein the compound of formula 1 is selected from among:
2-(1H-indazol-5-yl)amino-6-(2-isopropylimidazol-1-yl)-3-nitropyridine;
2-(1H-indazol-5-yl)amino-6-(isopropylamino)-3-nitropyridine;
2-(1H-indazol-5-yl)amino-6-(isobutylamino)-3-nitropyridine;
2-(1H-indazol-5-yl)amino-6-(4-hydroxypiperidino)-3-nitropyridine;
2-(1H-indazol-5-yl)amino-6-(piperazin-1-yl)-3-nitropyridine;
2-(1H-indazol-5-yl)amino-6-[(N-ethyl-2-hydroxyethyl)amino]-3-nitropyridine;
2-(1H-indazol-5-yl)amino-6-(t-butylamino)-3-nitropyridine;
2-(1H-indazol-6-yl)amino-6-[(2-pyridyl)ethylamino]-3-nitropyridine;
2-(1H-indazol-5-yl)amino-6-[(3-dimethylamino-2,2-dimethyl)propylamino]-3-nitropyridine;
2-(1H-indazol-5-yl)amino-6-(cyclopropylamino)-3-nitropyridine;
2-(1H-indazol-5-yl)amino-6-(4-methylpiperazin-1-yl)-3-nitropyridine;
2-(1H-indazol-5-yl)amino-6-(4-amino-n-butylamino)-3-nitropyridine;
and pharmaceutically acceptable salts thereof.

18. The method of claim 14, wherein the pharmaceutically acceptable salt is hydrochloride or methanesulfonate.

19. The method of 15, wherein the pharmaceutically acceptable salt is hydrochloride or methanesulfonate.

20. The method of claim 16, wherein the pharmaceutically acceptable salt is hydrochloride or methanesulfonate.

21. The method of claim 17, wherein the pharmaceutically acceptable salt is hydrochloride or methanesulfonate.

22. The composition of claim 5, wherein the pharmaceutically acceptable salt is hydrochloride or methanesulfonate.

23. The composition of claim 6, wherein the pharmaceutically acceptable salt is hydrochloride or methanesulfonate.

24. The composition according to claim 7, wherein the pharmaceutically acceptable salt is hydrochloride or methanesulfonate.

25. The composition of claim 4, wherein the compound of formula 1 is selected from among:
2-(1H-indazol-5-yl)amino-6-(2-methyl-4,5-dihydroimidazol-1-yl)amino-3-nitropyridine;
2-(1H-indazol-5-yl)amino-6-(2-methylimidazol-1-yl)-3-nitropyridine;
2-(1H-indazol-5-yl)amino-6-(2-isopropylimidazol-1-yl)-3-nitropyridine; and
2-(1H-indazol-5-yl)amino-6-(5-methylimidazol-1-yl)-3-nitropyridine;
or a pharmaceutically acceptable salt thereof.
